(19) United States
(12) Patent Application Publication
(10) Pub. No.: US 2023/0181236 A1
(43) Pub. Date: Jun. 15, 2023

(54) SYSTEM OF MEDICAL DEVICES AND METHOD FOR PERICARDIAL PUNCTURE

(71) Applicant: Boston Scientific Medical Device Limited, Ballybrit (IE)

(72) Inventors: Brock Miller, Toronto (CA); Matthew Gravett, Milton (CA)

(21) Appl. No.: 18/167,738

(22) Filed: Feb. 10, 2023

Related U.S. Application Data
(63) Continuation of application No. PCT/IB2021/057152, filed on Aug. 4, 2021.
(60) Provisional application No. 63/064,454, filed on Aug. 12, 2020.

(51) Int. Cl. A61B 18/14

(52) U.S. Cl. CPC A61B 18/14; A61B 2018/00363

(57) ABSTRACT

A system of medical devices includes a puncture device and an introducer. The puncture device extends from a puncture device proximal end to a puncture device distal end. The puncture device includes a radiofrequency puncture electrode at the puncture device distal end. The electrode is radiopaque. The introducer extends between an introducer proximal end and an introducer distal end. The introducer has a lumen extending therethrough from the introducer proximal end to the introducer distal end. The introducer includes at least a first radiopaque marker associated with the introducer distal end. The puncture device is advanceable through the lumen from the introducer proximal end towards the introducer distal end to position the puncture device in a puncture position in which the radiofrequency puncture electrode is proud of the introducer distal. When the puncture device is in the puncture position, the electrode is spaced distally from the first radiopaque marker.

### CROSS REFERENCE TO RELATED APPLICATION

This application is a continuation application of International Application No. PCT/IB2021/057152, filed Aug. 4, 2021, titled “SYSTEM OF MEDICAL DEVICES AND METHOD FOR PERICARDIAL PUNCTURE,” which claims priority to U.S. Provisional Application No. 63/064,454, filed Aug. 12, 2020, titled “SYSTEM OF MEDICAL DEVICES AND METHOD FOR PERICARDIAL PUNCTURE,” the entire disclosures of which are incorporated herein by reference.

### FIELD

This document relates to medical devices. More specifically, this document relates to systems of medical devices that can be used in pericardial puncture, and related methods.

### SUMMARY

The following summary is intended to introduce the reader to various aspects of the detailed description, but not to define or delimit any invention.

Systems of medical devices are disclosed. According to some aspects, a system of medical devices includes a puncture device and an introducer. The puncture device extends from a puncture device proximal end to a puncture device distal end. The puncture device includes a radiofrequency puncture electrode at the puncture device distal end. The radiofrequency puncture electrode is radiopaque. The introducer extends between an introducer proximal end and an introducer distal end. The introducer has a lumen extending therethrough from the introducer proximal end to the introducer distal end. The introducer includes at least a first radiopaque marker associated with the introducer distal end. The puncture device is advanceable through the lumen from the introducer proximal end towards the introducer distal end to position the puncture device in a puncture position in which the radiofrequency puncture is proud of the introducer distal end. When the puncture device is in the puncture position, the radiofrequency puncture electrode is spaced distally from the first radiopaque marker.

In some examples, when the puncture device is in the puncture position and the radiofrequency puncture electrode is spaced distally from the first radiopaque marker, there is a radiolucent section between the radiofrequency puncture electrode and the first radiopaque marker.

In some examples, the puncture device includes a core wire and an electrically insulative material on the core wire, and the radiofrequency puncture electrode includes an electrode puck received on the core wire and a domed electrode tip adjacent the electrode puck.

In some examples, the puncture device further includes a second radiopaque marker. The second radiopaque marker can include a radiopaque coil embedded in the electrically insulative material. The radiopaque coil can be positioned adjacent and proximally of the electrode puck, or spaced proximally from the electrode puck to provide the radiolucent section.

In some examples, the first radiopaque marker is at the introducer distal end. In some examples, the first radiopaque marker is proximate the introducer distal end.

In some examples, the introducer includes an introducer shaft, and the introducer shaft includes a polymeric sheathing and a radiopaque tube embedded in the polymeric sheathing. The radiopaque tube can provide the first radiopaque marker. The polymeric sheathing can extend to the introducer distal end, and the radiopaque tube can extend to a position shy of the introducer distal end, to provide the radiolucent section at the introducer distal end.

In some examples, the introducer includes an introducer shaft, and the first radiopaque marker includes a band in the introducer shaft. The band can be positioned shy of the introducer distal end, to provide the radiolucent section at the distal end.

Methods for pericardial puncture are also disclosed. According to some aspects, a method for pericardial puncture includes: a. advancing an introducer towards a pericardium; b. advancing a puncture device through the introducer towards the pericardium while viewing the puncture device and introducer under fluoroscopy; stopping advancement of the puncture device when, when viewed under fluoroscopy, a radiopaque radiofrequency puncture electrode is proud of a first radiopaque marker of the introducer; and d. puncturing the pericardium with the puncture device.

In some examples, step b. includes stopping advancement when, when viewed under fluoroscopy, a radiolucent section appears. In step b., the radiolucent section can appear between the radiopaque radiofrequency puncture electrode and the first radiopaque marker of the introducer, or between a radiopaque coil of the puncture device and the first radiopaque marker of the introducer.

In some examples, the appearance of a radiolucent section indicates that the puncture device is in a puncture position. In the puncture position, the radiofrequency puncture electrode of the puncture device can be proud of a distal end of the introducer, or the radiofrequency puncture electrode of the puncture device can be spaced distally from a distal end of the introducer.

In some examples, step d. includes delivering radiofrequency energy from the radiofrequency puncture electrode.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example system of medical devices;

FIG. 2 is a perspective view of the puncture device of the system of FIG. 1;

FIG. 3 is a cross-section taken along line 3-3 in FIG. 2;

FIG. 4 is a perspective view of the introducer of the system of FIG. 1;

FIG. 5 is a cross-section taken along line 5-5 in FIG. 4;

FIG. 6 is a cross-section taken along line 6-6 in FIG. 1;

FIG. 7 is a schematic view showing the appearance of the system of FIG. 1 under fluoroscopy, when the puncture device has not yet reached a puncture position;

FIG. 8 is a schematic view showing the appearance of the system of FIG. 1 under fluoroscopy, with the puncture device in a puncture position;

FIG. 9 is a cross section similar to that of FIG. 6, of an alternative system of medical devices;

FIG. 10 is a schematic view showing the appearance of the system of FIG. 9 under fluoroscopy, when the puncture device has not yet reached a puncture position;

FIG. 11 is a schematic view showing the appearance of the system of FIG. 9 under fluoroscopy, with the puncture device in a puncture position;

FIG. 12 is a cross section similar to that of FIG. 6, of another alternative system of medical devices;

FIG. 13 is a schematic view showing the appearance of the system of FIG. 12 under fluoroscopy, when the puncture device has not yet reached a puncture position;

FIG. 14 is a schematic view showing the appearance of the system of FIG. 12 under fluoroscopy, with the puncture device in a puncture position;

FIG. 15 is a schematic view showing the appearance of the system of FIG. 12 under fluoroscopy, with the puncture device advanced beyond a puncture position;

FIG. 16 is a cross section similar to that of FIG. 6, of another alternative system of medical devices;

FIG. 17 is a schematic view showing the appearance of the system of FIG. 16 under fluoroscopy, when the puncture device is in a first puncture position; and

FIG. 18 is a schematic view showing the appearance of the system of FIG. 16 under fluoroscopy, with the puncture device in a second puncture position.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein is a system of medical devices that includes an introducer and a puncture device. The system is configured to provide the user with an indication of the relative position of a radiofrequency puncture electrode of the puncture device and the distal end of the introducer, even if the radio frequency puncture electrode and the distal end of the introducer are not visible with the naked eye (i.e. not visible when the system is in use and the radiofrequency puncture electrode and the distal end of the introducer are inside the body). More specifically, the system can provide the user with an indication that the puncture device is in a puncture position -i.e. positioned with the radiofrequency puncture electrode proud of the distal end of the introducer, so that radiofrequency energy can be delivered from the radiofrequency puncture electrode to puncture tissue. As will be described in detail below, in order to provide the user with an indication that the puncture device is in the puncture position, the system can be configured so that when viewed under fluoroscopy, a radiolucent gap appears between the radiofrequency puncture electrode and the introducer when the puncture device is in the puncture position.

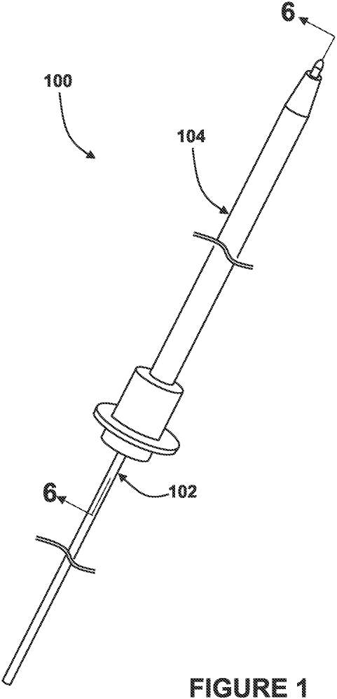

Referring now to FIG. 1, an example system 100 of medical devices is shown. The system 100 generally includes a puncture device 102 and an introducer 104. The puncture device 102 is receivable in and advanceable through the introducer 104. The introducer 104 can serve to atraumatically guide the puncture device 102 towards a target location in a patient’s body (e.g. the heart), and the puncture device 102 can then puncture the target location (e.g. puncture the pericardium).

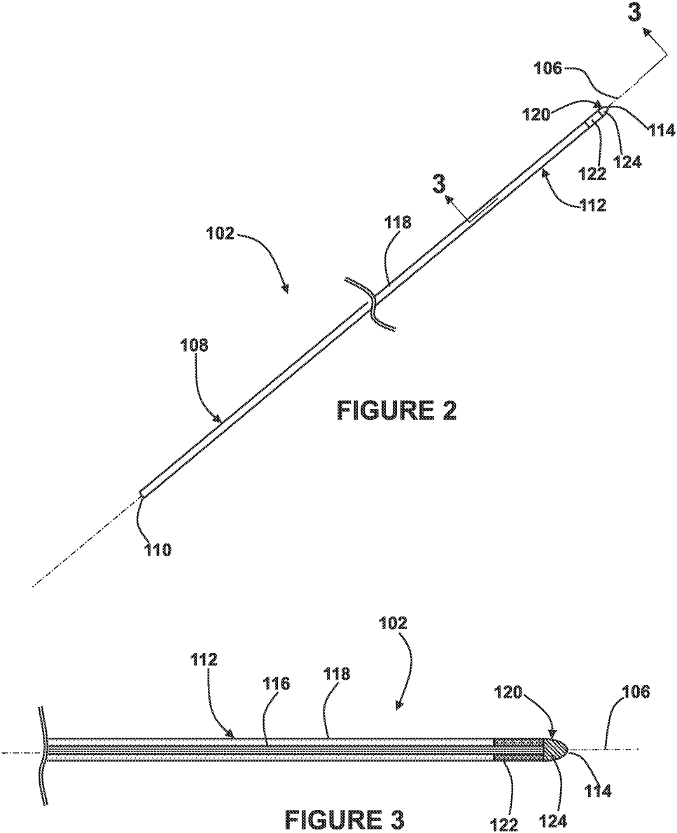

Referring now to FIGS. 2 and 3, the puncture device 102 extends along a longitudinal axis 106 and has a proximal portion 108 (also referred to herein as a ‘puncture device proximal portion’), which defines a proximal end 110 (also referred to herein as a ‘puncture device proximal end’), and a distal portion 112 (also referred to herein as a ‘puncture device distal portion’), which defines a distal end 114 (also referred to herein as a ‘puncture device distal end). The puncture device 102 has a length (also referred to herein as a ‘length length’) between the proximal end 110 and the distal end 114.

In the example shown, the puncture device 102 is a radiofrequency (RF) puncture device, and includes a core wire 116 and an electrically insulative material 118 on the core wire 116. An RF puncture electrode 120 is at the distal end 114 of the puncture device 102. In the example shown, the RF puncture electrode 120 is radiopaque. More specifically, in the example shown, the RF puncture electrode includes a radiopaque electrode puck 122 received on the core wire 116, and a radiopaque domed electrode tip 124 adjacent and distal to the electrode puck 122. The electrode puck 122 and the electrode tip 124 can both be fabricated from or can include an electrically conductive radiopaque material such as platinum-iridium.

The proximal portion 108 of the puncture device 102 can be connected to an RF generator (not shown, which may in turn be connected to one or more grounding pads), so that RF energy can be delivered from the RF generator to the RF puncture electrode 120 via the core wire 116. When the RF puncture electrode 120 is in contact with a tissue (e.g. the pericardium) and RF energy is delivered to the RF puncture electrode 122, the RF energy causes puncture of the tissue.

In the example shown, the puncture device 102 is relatively flexible, and can also serve as a guidewire.

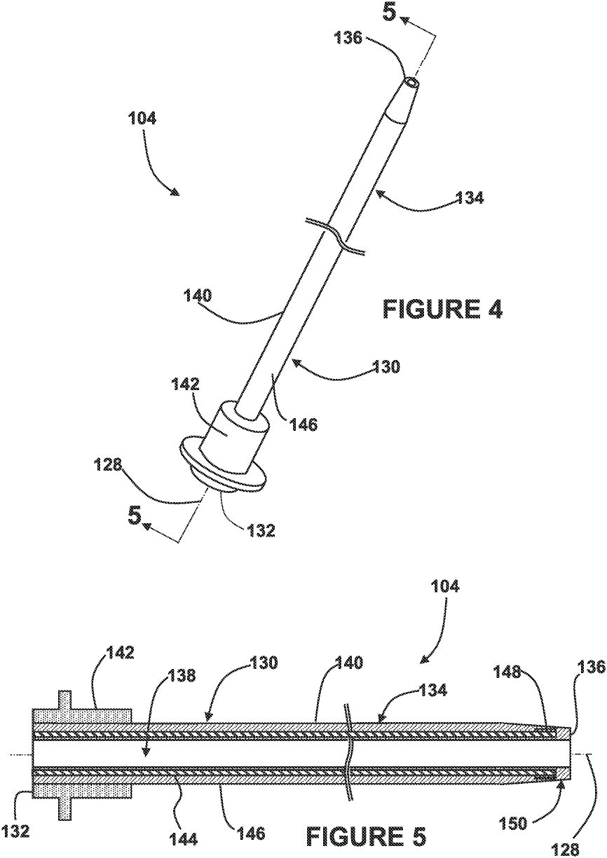

Referring now to FIGS. 4 and 5, the introducer 104 extends along a longitudinal axis 128 and has a proximal portion 130 (also referred to herein as an ‘introducer proximal portion’), which defines a proximal end 132 (also referred to herein as an ‘introducer proximal end’), and a distal portion 134 (also referred to herein as an ‘introducer distal portion’), which defines a distal end 136 (also referred to herein as an ‘introducer distal end). The introducer 104 has a length (also referred to herein as an ‘introducer length’) between the proximal end 132 and the distal end 136. A lumen 138 (shown in FIG. 5) extends through the introducer 104 from the proximal end 132 to the distal end 136, for receiving the puncture device 102.

Referring still to FIGS. 4 and 5, in the example shown, the introducer 104 includes a shaft 140 (also referred to herein as an ‘introducer shaft’) and a hub 142 (also referred to herein as an ‘introducer hub’). The hub 142 defines the introducer proximal end 132 while the shaft 140 defines the introducer distal end 136. The hub 142 may include various features, such as fluid ports and hemostatic valves, etc. (not shown). In use, the hub 142 may be grasped and manipulated by the user, while the distal portion 134 is directed to a target site within a patient’s body (e.g. the heart). The distal end 136 is blunt, to avoid damaging tissue in contact with the distal end 136.

As shown in FIG. 5, in the example shown, the shaft 140 includes a metallic tube 144 (e.g. a stainless steel hypotube) that reinforces the introducer 104, and a polymeric sheathing 146 (e.g. a high-density polyethylene sheathing) on the tube 144. The polymeric sheathing 146 is slightly longer than the metallic tube 144. That is, the polymeric sheathing 146 extends to the introducer distal end 136, while the metallic tube 144 extends to a position shy of the introducer distal end 136. For example, the polymeric sheathing 146 can be longer than the metallic tube 144 by about 1 mm, or up to about 5 mm.

Referring still to FIG. 5, the introducer 104 includes at least a first radiopaque marker 148 that is associated with the distal end 136 of the introducer 104. The term “associated with” means that the first radiopaque marker 148 is right at the distal end 136 of the introducer 136, or is proximate but spaced from the distal end 136 (i.e. about 1 mm from the distal end 136 or within about 5 mm of the distal end 136). In the example shown, the first radiopaque marker 148 is in the form of a band of radiopaque material (e.g. platinum or platinum-iridium) that is in the introducer shaft 140. More specifically, the band is received on the metallic tube 144, and is embedded in the polymeric sheathing 146. The band is at the end of the metallic tube 144, and as the polymeric sheathing 146 is longer than the metallic tube 144, the band is spaced from the distal end 136 of the introducer 104. Accordingly, the distalmost section of the polymeric sheathing 146 provides a radiolucent section 150 at the distal end 136 of the introducer 104. In alternative examples, the radiopaque marker can be in the form of a section of the polymeric sheathing that is filled with a radiopaque filler (e.g. barium sulphate).

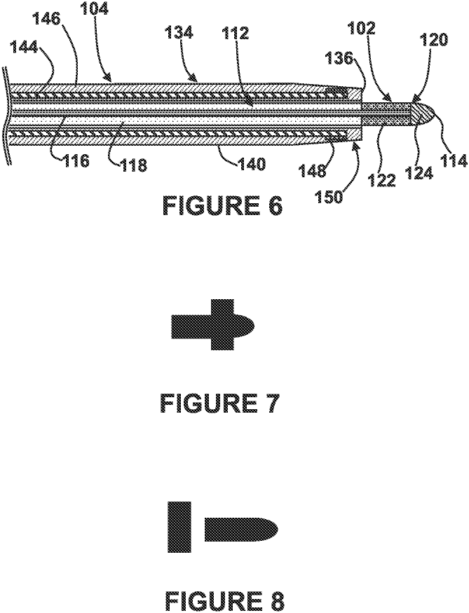

Referring now to FIG. 6, the puncture device 102 is advanceable through the lumen 138 (not labelled in FIG. 6) of the introducer 104, from the introducer proximal end 132 (not shown in FIG. 6) towards the introducer distal end 136, to position the puncture device 102 in a “puncture position”. In the puncture position, the RF puncture electrode 120 is proud of the introducer distal end 136, so that it can contact and delivery RF energy to a target tissue (e.g. the pericardium), while still being supported by the introducer 104. In the example shown, in the puncture position, the RF puncture electrode 120 is just proud of the introducer distal end (i.e. the proximal-most point of the electrode is 120 aligned with the distal end 136 of the introducer 104). In alternative examples, as will be described below, in the puncture position, the RF puncture electrode 120 can be proud of the introducer distal end 136 by a predetermined distance (i.e. the RF puncture electrode 120 can be spaced distally from the distal end 136 of the introducer 104). In use, when the puncture device 102 is in the puncture position, the electrode 120 is spaced distally from the first radiopaque marker 148.

Due to the configuration of the electrode 120 (which is radiopaque) and the first radiopaque marker 148, when the puncture device is in the puncture position (i.e. with the electrode 120 spaced distally from the first radiopaque marker 148), the radiolucent section 150 is between the electrode 120 and the first radiopaque maker 148. Accordingly, in use, the system can be viewed under fluoroscopy, and the appearance of the radiolucent section 150 can indicate that the puncture device 102 is in the puncture position. More specifically, in use, the introducer 104 can be advanced towards the pericardium (e.g. via the subxiphoid approach, optionally with a stylet in the lumen 138 to prevent tissue coring). The puncture device 102 can then be advanced through the introducer 104 towards the pericardium, while viewing the introducer 104 and puncture device 102 under fluoroscopy. As the electrode 102 approaches the puncture position - i.e. when the distal 114 end of the puncture device 102 is flush with the distal end 136 of the introducer 104 - the fluoroscopic view will appear as shown in FIG. 7. As advancement of the puncture device 102 continues, the electrode 120 will advance beyond the radiopaque marker 148, and the radiolucent section 150 will appear in the fluoroscopic view between the electrode 120 and the first radiopaque marker 148, as shown in FIG. 8. The appearance of the radiolucent section 150 between the electrode 120 and the first radiopaque marker 148 indicates that the puncture device 102 is in the puncture position. When the radiolucent section 150 appears between the electrode 120 and the first radiopaque marker 148 - i.e. when the electrode 120 is proud of the radiopaque marker 148 and the puncture device is in the puncture position - advancement of the puncture device 102 can be stopped.

Once the puncture device is in the puncture position, the pericardium can be punctured with the puncture device 102. That is, RF energy can be delivered from the RF puncture electrode 120, to puncture the pericardium. The puncture device 102 can then optionally be advanced into the pericardial space, and can serve as a guidewire in subsequent steps of the medical procedure.

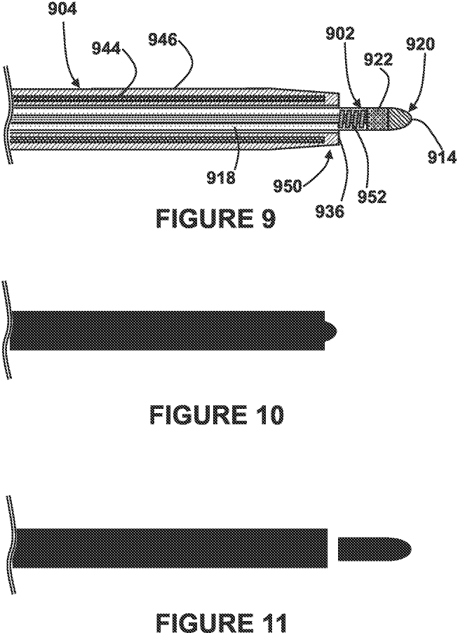

Referring now to FIG. 9, another example of an introducer and a puncture device is shown. In FIG. 9, features that are like those of FIGS. 1 to 8 will be referred to with like reference numerals, incremented by 800.

The introducer 904 is similar to the introducer 104; however, in the introducer 904, the entire metallic tube 944 is radiopaque and serves as the radiopaque marker of the introducer 904. Similarly to the introducer 104, the polymeric sheathing 946 extends to the introducer distal end 936, and the tube 944 extends to a position shy of the introducer distal end 936, to yield a radiolucent section 950 at the introducer distal end 936.

The puncture device 902 is similar to the puncture device 102; however, the puncture device includes an additional radiopaque marker 952 (also referred to herein as a “second radiopaque marker”). The additional radiopaque marker 952 is in the form of a radiopaque coil that is embedded in the electrically insulative material 918, and is positioned adjacent and proximally of the electrode 920 (i.e. proximally of and adjacent the electrode puck 922 of the electrode 920).

FIG. 9 shows the puncture device 902 in the puncture position. In the puncture position, the RF puncture electrode 920 is spaced distally from the introducer distal end 936. More specifically, the RF puncture electrode 920 is spaced distally from the introducer distal end 936 by a predetermined distance that is equal to the length of the additional radiopaque marker 952.

The puncture device 902 and introducer 904 can be used in a similar fashion to those of FIGS. 1 to 8. That is, the puncture device 902 can be advanced through the introducer 904 towards the pericardium, while viewing the introducer 904 and puncture device 902 under fluoroscopy. As the electrode 902 approaches the puncture position - e.g. when the distal 914 end of the puncture device 902 is flush with the distal end 936 of the introducer 904 - the fluoroscopic view will appear as shown in FIG. 10. As advancement of the puncture device 904 continues, the electrode 920 will advance beyond the metallic tube 944, then the second radiopaque marker 952 will advance beyond the metallic tube 944, and then the radiolucent section 950 will appear in the fluoroscopic view, between the second radiopaque marker 952 and the metallic tube 944, as shown in FIG. 11. The appearance of the radiolucent section 950 indicates that the puncture device 902 is in the puncture position. When the radiolucent section 950 appears, advancement of the puncture device 102 can be stopped.

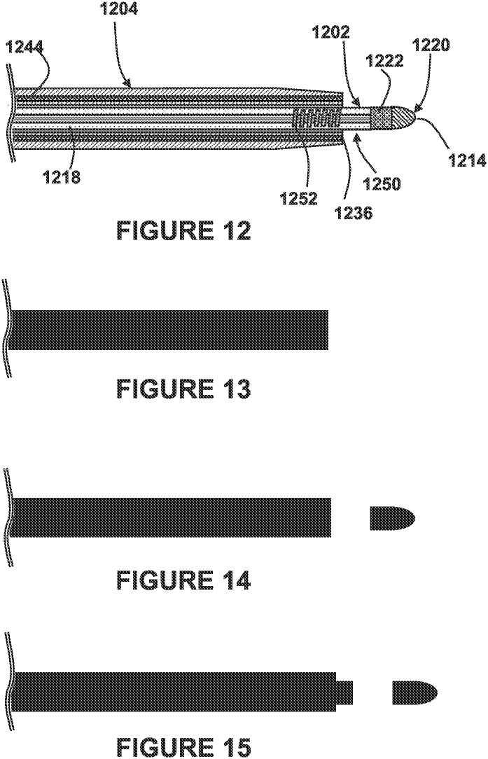

Referring now to FIG. 12, another example of an introducer and a puncture device is shown. In FIG. 12, features that are like those of FIGS. 1 to 8 will be referred to with like reference numerals, incremented by 1100.

The introducer 1204 is similar to the introducer 904 of FIG. 9; however, the radiopaque metallic tube 1244 extends right to the distal end 1236 of the introducer.

The puncture device 1202 is similar to the puncture device 902; however, the additional radiopaque marker 1252 (also referred to herein as a “second radiopaque marker”) is in the form of a radiopaque coil that is embedded in the electrically insulative material 1218, and is spaced proximally from the electrode 1220 (i.e. from the electrode puck 1222 of the electrode 1220), to provide a radiolucent section 1250 between the electrode 1220 and the radiopaque marker 1252. In use, when viewed under fluoroscopy, the appearance of the radiolucent section 1250 indicates that the puncture device 1202 is in the puncture position.

The puncture device 1202 and introducer 1204 can be used in a similar fashion to those of FIG. 9. That is, the puncture device 1202 can be advanced through the introducer 1204 towards the pericardium, while viewing the introducer 1204 and puncture device 1202 under fluoroscopy. As the electrode 1220 approaches the puncture position - e.g. when the distal 1214 end of the puncture device 1202 is flush with the distal end 1236 of the introducer 1204 - the fluoroscopic view will appear as shown in FIG. 13. As advancement of the puncture device 1202 continues, the electrode 1220 will advance beyond the metallic tube 1244, and the radiolucent section 1250 will appear in the fluoroscopic view between the electrode 1220 and the tube 1244, as shown in FIG. 14. Appearance of the radiolucent section 1250 indicates that the puncture device 1202 is in the puncture position. If advancement continues, the second radiopaque marker 1252 will appear in the fluoroscopic view, as shown in FIG. 15. Appearance of the second radiopaque marker 1252 in the fluoroscopic view indicates that the puncture device 1202 has advanced too far. The puncture device can then be retracted slightly, until the second radiopaque marker 1252 is no longer visible (i.e. until the fluoroscopic view again appears as shown in FIG. 14). When the fluoroscopic view appears as shown in FIG. 14, advancement of the puncture device 1202 can be stopped.

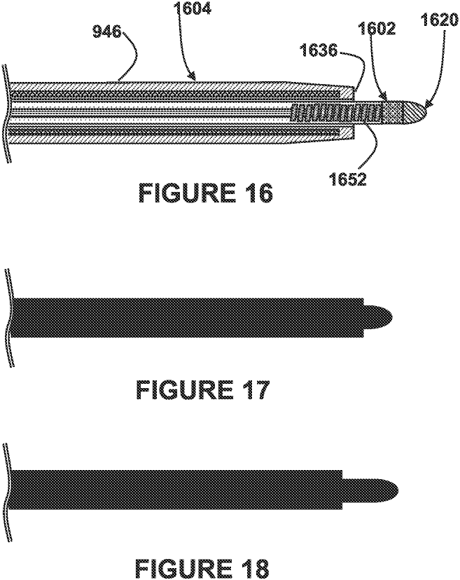

Referring now to FIG. 16, another example of an introducer and a puncture device is shown. In FIG. 16, features that are like those of FIGS. 1 to 8 will be referred to with like reference numerals, incremented by 1500.

The introducer 1604 is the same as the introducer 904 of FIG. 9.

The puncture device 1602 is similar to the puncture device 902; however, the additional radiopaque marker 1652 (also referred to herein as a “second radiopaque marker”) is relatively long.

The puncture device 1602 and introducer 1604 can be used in a similar fashion to those of FIG. 9; however the puncture device 1602 and introducer 1604 are simply configured to provide an indication that the electrode 1620 is proud of the introducer distal end 1636, and do not rely on a radiolucent section. That is, the puncture device 1602 can be advanced through the introducer 1604 towards the pericardium, while viewing the introducer 1604 and puncture device 1602 under fluoroscopy. Advancement of the puncture device 1602 can be stopped when the puncture device 1602 is visible in the fluoroscopic view, whether as shown in FIG. 17 or as shown in FIG. 18

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be revisited.

We claim:

1. A system of medical devices, comprising:
a puncture device extending from a puncture device proximal end to a puncture device distal end, wherein the puncture device comprises a radiofrequency puncture electrode at the puncture device distal end, and wherein the radiofrequency puncture electrode is radiopaque; and
an introducer extending between an introducer proximal end and an introducer distal end, wherein the introducer has a lumen extending therethrough from the introducer proximal end to the introducer distal end, and wherein the introducer comprises at least a first radiopaque marker associated with the introducer distal end;
wherein the puncture device is advanceable through the lumen from the introducer proximal end towards the introducer distal end to position the puncture device in a puncture position in which the radiofrequency puncture electrode is proud of the introducer distal end; and
wherein when the puncture device is in the puncture position, the radiofrequency puncture electrode is spaced distally from the first radiopaque marker.

2. The system of claim 1, wherein when the puncture device is in the puncture position and the radiofrequency puncture electrode is spaced distally from the first radiopaque marker, there is a radiolucent section between the radiofrequency puncture electrode and the first radiopaque marker.

3. The system of claim 2, wherein the puncture device comprises a core wire and an electrically insulative material on the core wire, and
the radiofrequency puncture electrode comprises an electrode puck received on the core wire and a domed electrode tip adjacent the electrode puck.

4. The system of claim 3, wherein the puncture device further comprises a second radiopaque marker.

5. The system of claim 4, wherein the second radiopaque marker comprises radiopaque coil embedded in the electrically insulative material.

6. The system of claim 5, wherein the radiopaque coil is positioned adjacent and proximally of the electrode puck.

7. The system of claim 5, wherein the radiopaque coil is spaced proximally from the electrode puck to provide the radiolucent section.

8. The system of claim 1, wherein the first radiopaque marker is at the introducer distal end.

9. The system of claim 1, wherein the first radiopaque marker is proximate the introducer distal end.

10. The system of claim 1, wherein the introducer comprises an introducer shaft, and the first radiopaque marker comprises a band in the introducer shaft.

11. The system of claim 10, wherein the band is positioned shy of the introducer distal end, to yield a radiolucent section at the introducer distal end.

12. A system of medical devices, comprising:
a puncture device extending from a puncture device proximal end to a puncture device distal end, wherein the puncture device comprises a radiofrequency puncture electrode at the puncture device distal end, and wherein the radiofrequency puncture electrode is radiopaque; and
an introducer extending between an introducer proximal end and an introducer distal end, wherein the introducer has a lumen extending therethrough from the introducer proximal end to the introducer distal end, and wherein the introducer comprises an introducer shaft, and the introducer shaft comprises a polymeric sheathing and a radiopaque tube embedded in the polymeric sheathing;
wherein the puncture device is advanceable through the lumen from the introducer proximal end towards the introducer distal end to position the puncture device in a puncture position in which the radiofrequency puncture electrode is proud of the introducer distal end; and
wherein when the puncture device is in the puncture position, the radiofrequency puncture electrode is spaced distally from the radiopaque tube.

13. The system of claim 12, wherein the radiopaque tube extends to the introducer distal end.

14. The system of claim 12, wherein the polymeric sheathing extends to the introducer distal end, and the radiopaque tube extends to a position shy of the introducer distal end, to provide a radiolucent section at the introducer distal end.

15. A method for pericardial puncture, comprising:
a. advancing an introducer towards a pericardium;
b. advancing a puncture device through the introducer towards the pericardium while viewing the puncture device and introducer under fluoroscopy,
c. stopping advancement of the puncture device when, when viewed under fluoroscopy, a radiopaque radiofrequency puncture electrode is proud of a first radiopaque marker of the introducer;
d. puncturing the pericardium with the puncture device.

16. The method of claim 15, wherein step c. comprises stopping advancement when, when viewed under fluoroscopy, a radiolucent section appears.

17. The method of claim 16, wherein in step c. the radiolucent section appears between the radiopaque radiofrequency puncture electrode and the first radiopaque marker of the introducer.

18. The method of claim 15, wherein in step c., the radiolucent section appears between a radiopaque coil of the puncture device and the first radiopaque marker of the introducer.

19. The method of claim 16, wherein the appearance of the radiolucent section indicates that the puncture device is in a puncture position.

20. The method of claim 19, wherein in the puncture position, the radiofrequency puncture electrode of the puncture device is proud of a distal end of the introducer.
